SEQUENCE LISTING (24 sequences)

SEQ ID NO: 1 - DNA, 10 bp, Artificial
  Human example sequence
accgtaccgg

SEQ ID NO: 2 - DNA, 12 bp, Homo sapiens
cctttggggggt

SEQ ID NO: 3 - DNA, 12 bp, Homo sapiens
cctctgtggggt

SEQ ID NO: 4 - DNA, 11 bp, Homo sapiens
attggagtttc

SEQ ID NO: 5 - DNA, 11 bp, Homo sapiens
tttggggtttc

SEQ ID NO: 6 - DNA, 10 bp, Homo sapiens
gaggggtatc

SEQ ID NO: 7 - DNA, 10 bp, Homo sapiens
tgggggtatc

SEQ ID NO: 8 - DNA, 10 bp, Homo sapiens
ggggtatctg

SEQ ID NO: 9 - DNA, 10 bp, Homo sapiens
ggggtatctg

SEQ ID NO: 10 - DNA, 12 bp, Homo sapiens
tgacctttgggg

SEQ ID NO: 11 - DNA, 14 bp, Homo sapiens
gccgcgggccgcac

SEQ ID NO: 12 - DNA, 14 bp, Homo sapiens
  misc_feature (5)..(5): n is a, c, g, or t
gccgngggccgcac

SEQ ID NO: 13 - DNA, 10 bp, Homo sapiens
tgtgtgtgtc

SEQ ID NO: 14 - DNA, 10 bp, Homo sapiens
tgtgtgtatc

SEQ ID NO: 15 - DNA, 10 bp, Homo sapiens
tactaaaaaa

SEQ ID NO: 16 - DNA, 10 bp, Homo sapiens
tacaaaaaaa

SEQ ID NO: 17 - DNA, 10 bp, Homo sapiens
aataataata

SEQ ID NO: 18 - DNA, 10 bp, Homo sapiens
aataaaaata

SEQ ID NO: 19 - DNA, 13 bp, Homo sapiens
aataataaataaa

SEQ ID NO: 20 - DNA, 13 bp, Homo sapiens
aataaaaaataaa

SEQ ID NO: 21 - DNA, 10 bp, Homo sapiens
aaaaataata

SEQ ID NO: 22 - DNA, 10 bp, Homo sapiens
aataataata

SEQ ID NO: 23 - DNA, 12 bp, Homo sapiens
aaggcacgggcg

SEQ ID NO: 24 - DNA, 10 bp, Homo sapiens
tactaaaaaa

(19) United States
(12) Patent Application Publication
(10) Pub. No.: US 2009/0164135 A1
(43) Pub. Date: Jun. 25, 2009

(54) QUATERNIONIC ALGEBRA APPROACH TO DNA AND RNA TANDEM REPEAT DETECTION

(76) Inventors: Andrzej K. BRODZIK, Arlington, MA (US); Olivia J. Peters, Leesburg, VA (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/962,930

(22) Filed: Dec. 21, 2007

(51) Int. Cl. G01N 33/48; G06F 19/00

(52) U.S. Cl. 702/20

(57) ABSTRACT

A method of detecting and outputting tandem repeats in a sequence of symbols comprising a) mapping the symbols to quaternions; b) constructing a Quaternionic Periodicity Transform (QPT); c) computing the QPT of the sequence to determine the tandem repeats of the sequence; d) post-processing of the QPT; e) outputting a list of tandem repeats obtained from step d) to a computer's memory. In embodiments, the sequence of symbols is a sequence of letters representing nucleotides in a DNA or RNA sequence.

### BACKGROUND OF THE INVENTION

1. Field of the Invention

The present invention relates to methods of detecting periodicities in a sequence of symbols. The invention further relates to detecting tandem repeats in a sequence of DNA or RNA.

2. Background Art

DNA or RNA data contains symbol sequences that do not exhibit an obvious order and sequences made up of symbol patterns that repeat periodically. The latter sequences arouse interest because they are unexpected and because they provide a convenient visual and numerical reference. DNA repeats can also, in general, be classified, studied and endowed with biological significance easier than random assemblies of symbols. In molecular biology research DNA repeats are important, as they can be associated with specific biological phenomena, e.g., evolutionary transmission of information, and be used as biomarkers for genetic diseases.

Many different types of repetitions occur in the DNA data. At the most general level, repetitive patterns can be divided into tandem repeats, dispersed repeats and structural repeats. A tandem repeat consists of two or more adjacent copies of an arbitrary sequence of DNA symbols. The length of the sequence typically varies from a few to a few hundred of bases. Dispersed repeats consist of two or more non-adjacent copies of an arbitrary sequence. Such sequences are often of a greater length than tandem repeat patterns. Both tandem and dispersed repeats occur mainly in non-conserved regions of genomes. Dispersed repeats alone are estimated to comprise about one-half of the human genome (Lander et al., 2001). Structural repeats are defined by an over-representation of subsets of DNA sequences of a certain length. They are indicative, for example, of encoding for amino acids in the transcribable sections of DNA and of the helical structure of the DNA double strand.

The best known of the DNA repeats are the tandem repeats. Tandem repeats encode information about the structure and function of DNA and thereby play a key role in a number of applications. One of the most important of these applications is the diagnosis of genetic disorders. Occurrence of tandem repeats and tandem repeat changes in the human genome have been associated with Huntington's disease (HDCRG, 1993), myotonic dystrophy (Fu et al., 1992), Friedreich's ataxia (Campuzano et al., 1996), multiple sclerosis (Guerini et al., 2003), Alzheimer's disease (Licastro et al., 2003), schizophrenia (Brzustowicz et al., 2000) and cancer (Sidransky, 1997). In those cases occurrence of repetitions in specific parts of the genome indicates pathology. In other key applications, such as DNA forensics (Butler, 2003) and reconstruction of human evolutionary history (Tishkoff et al., 2000), tandem repeats allows the differentiation between individuals, or between geographically and temporarily separated populations.

Furthermore, as genetic markers can also be used in microbial forensics, tandem repeat analysis provides a powerful tool for investigation of infectious disease outbreaks (Cummings, 2002). Since the availability of the genomic data is relatively recent, and research that is able to fully take advantage of this data is just beginning, the number of tandem repeat applications is likely to grow even further. For these reasons, the design of ever more sensitive and efficient tools for DNA repeat analysis is a task of a considerable importance.

The methods used to detect DNA repeats can be classified as either stochastic or deterministic. A review of some of the more popular techniques was recently given in (Krishnan, 2004). In general, stochastic methods are preferred over deterministic ones, in part because they are thought to be better able to differentiate between significant and insignificant repeats. In practice, this advantage might not be fully realized, as the concepts of statistical and biological significance often diverge (Stolovitzky and Califano, 1998).

In contrast to stochastic techniques, deterministic and, in particular, algebraic methods have the advantage of being able, at least in principle, to detect all repeats, and of having computational complexity that is independent of the data. Among the deterministic approaches, many rely on spectral analysis of the data. The analysis is typically based either on Fourier (Anastassiou, 2001), Walsh (Tavare and Giddings, 1989) or wavelet transform (Arneodo et al., 1998) space processing. These methods target mainly structural and dispersed repeats. More recently, a time-domain method, called the periodicity transform has been introduced (Buchner and Janjarasjitt, 2003). Unlike the spectral methods, periodicity transform is well suited for tandem repeat detection, and it is also more computationally efficient. Despite these advantages, a wider use of periodicity transform has been limited, however, by several deficiencies that were not resolved in prior formulations. These include: (i) symbol bias that is inherent in the mapping of DNA symbols to complex numbers and which results in missed detections of some repetitive structures; (ii) lack of an appropriate post-processing stage that would remove redundant and insignificant repeats and (iii) absence of a strategy for identification of indels.

### BRIEF SUMMARY OF THE INVENTION

The present invention provides a method of detecting and outputting tandem repeats in a sequence of symbols comprising mapping the symbols to quaternions; constructing a Quaternionic Periodicity Transform (QPT); computing the QPT of the sequence to determine the tandem repeats of the sequence; post-processing of the QPT; and outputting the list of tandem repeats obtained to a computer's memory.

The present invention also provides a method of detecting and outputting tandem repeats in a sequence of symbols comprising mapping the symbols to quaternions to obtain a numerical sequence; applying the periodicity transform on a subsequence of the numerical sequence at each position of the sequence to generate the closest periodic sequence to the subsequence; repeating this step for each portion of the sequence and selecting repeats that satisfy pre-determined thresholds; removing from the selected repeats those that are either short, ambiguous, or contain a high number of errors; outputting sequence repeats, the number of repeats, the positions of the repeats and the length of the repeats to a computer's memory; and displaying the results in a graph.

### BRIEF DESCRIPTION OF THE DRAWINGS/FIGURES

FIG. 1 is an analysis of approximate tandem repeats in human microsatellite sequence M65145: raw periodicity transform (top) and post-processed periodicity transform (bottom). QPT parameters were N=1085, 1≦P≦12, T=0.85, R=2, minimum repeat length=12.

FIG. 2 is an analysis of approximate tandem repeats in the human frataxin gene, sequence U43748. Plots: raw periodicity transform (top) and post-processed periodicity transform (bottom). QPT parameters: N=2520, 1≦P≦48, T=0.9, R̅=2, minimum repeat length=12.

FIG. 3 is a plot of the tandem repeat detection run times of the QPT algorithm for subsets of Arabidopsis thaliana, chromosome 2.

### DETAILED DESCRIPTION OF THE INVENTION

### Definitions

The term “sequence of symbols,” as used herein, refers to a list of representations of objects. Typically the objects are represented by letters or numbers. The letters or numbers usually are abbreviations for the full names of the objects in the list. The length of the sequence of repeated symbols typically varies from a few symbols to a few hundred symbols; the repeated sequences are part of much longer sequences, of the order of millions or billions of symbols. The invention is capable of detecting tandem repeats in a sequence of virtually any length. The number of unique symbols, e.g., letters, present in the list can vary also, but typically four unique symbols are present in the sequence of symbols. An example of a sequence of symbols used in the present invention is a list of symbols that represent the nucleotides present in a molecule of deoxyribonucleic acid (“DNA”) or ribonucleic acid (“RNA”). DNA and RNA are both polymers of subunits; the subunits are called nucleotides. Nucleotides contain three characteristic components: (1) a nitrogenous base, (2) a pentose sugar, and (3) phosphoric acid, so that the base is covalently joined in an N-glycosyl linkage to carbon atom 1′ of the pentose and the phosphoric acid is esterified to carbon 5′.

The nitrogenous bases are derivatives of two parent heterocyclic compounds, pyrimidine and purine. DNA contains two principal pyrimidine bases, cytosine and thymine and two principal purine bases, adenine and guanine. RNAs also contain two principal purine bases, adenine and guanine. The single important difference in the bases of DNA and RNA is that thymine is a principal pyrimidine in DNAs but does not occur often in RNAs; conversely, uracil is a principal pyrimidine in RNAs but occurs only rarely in DNAs. Another major difference between DNA and RNA is that the pentose sugar in DNA is 2-deoxy-ribose, whereas in RNA, the 2-deoxy-ribose is replaced by ribose.

Nucleotides are linked together in a DNA or RNA molecule via phosphodiester bonds; thus, the backbone of the DNA or RNA strand is made from alternating phosphate and sugar residues. One strand of DNA or RNA associates with another strand of DNA or RNA through hydrogen bonding of the nitrogenous bases. The two strands of DNA or RNA therefore are antiparallel and form a double helix.

Generally a DNA or RNA molecule is identified through the number and sequence of nucleotides present on one of the DNA or RNA strands. Typically, the nucleotides are given a single letter designation, such as C, T, A, or G in the case of DNA or C, U, A or Gin the case of RNA.

As used herein the term “tandem repeat” refers to the presence of two or more adjacent copies of an arbitrary sequence of symbols in a sequence of symbols. In embodiments of the present invention, one or more anomalous symbols may appear in between the tandem repeats. The term “anomalous” refers to symbols that do not repeat in the pattern.

As used herein, the term “mapping” refers to associating each symbol in a sequence of symbols with a quaternion, i.e., a four dimensional hypercomplex number.

The term “quaternion,” as used herein, refers to a four-dimensional hypercomplex number. A quaternion is a number of the form q=a+bi+cj+dk, where a, b, c, d are real numbers, and i, j, k are symbols defined by the following set of rules:

i²=j²=k²=−1,

ij=−ji=k,

jk=−kj=i,

ki=−ik=j.

As used herein, the term Quaternionic Periodicity Transform (“QPT”) refers to a computational method for determining patterns in nonorthogonal subspaces such as the periodic subspaces Sp. Thus the order in which the projections occur effects the decomposition, and the Periodicity Transform does not in general provide a unique representation. Once the succession of the projections is specified, however, then the answer is unique.

The Periodicity Transform searches for the best periodic characterization of the length N signal x. The underlying technique is to project x onto some periodic subspace Sp. This periodicity is then removed from x leaving the residual r stripped of its p-periodicities. Both the projection x and the residual r may contain other periodicities, and so may be decomposed into other q-periodic components by further projection onto Sq. The intended goal of the decomposition, the amount of computational resources available, and the measure of “goodness-of-fit” all influence the algorithm.

As used herein, the term “tandem repeat with symbol error(s)” refers to the presence of two or more adjacent copies of an arbitrary sequence of symbols in a sequence of symbols, where the copies of the arbitrary sequence of symbols are not exact. For example, a tandem repeat containing two patterns may contain a symbol in one of the patterns that is different from the corresponding symbol in the adjacent pattern (ACCGT ACCGG). In embodiments of the invention, the user can determine a threshold value for symbol errors.

The term “alias tandem repeat” refers to a repeat occurring at a period that is a multiple of a fundamental repeat. For example, in the tandem repeat GTGTGTGT, the string GTGT would be considered an alias of GT.

The term “exact tandem repeat” as used herein refers to tandem repeats where the arbitrary sequence of symbols is precisely repeated in adjacent copies, with no symbol errors.

As used herein, the term “approximate tandem repeat” refers to a tandem repeat that is not exact. In an approximate tandem repeat, for example, one, two, three, etc. of the symbols are different from the copy that is adjacent to it.

As the term is used herein, “nested tandem repeat” refers to a tandem repeat that occurs within a larger tandem repeat.

The term “indel,” as used herein, refers to an insertion or deletion of a symbol in the adjacent repeat. For example, one or more copies of the tandem repeat may contain the symbols AGGTC, whereas the second or third, etc. copy of the tandem repeat may contain AGGC. The absence of the T in the sequence is referred to as an “indel.” Using the same example of AGGTC, this term also refers to a copy that is AGGXTC, where “X” is an inserted “extra” nucleotide not present in the first copy.

Introduction

The present invention provides a new method of identifying tandem repeats in DNA and RNA. Tandem repeats encode information about the structure and function of DNA and thereby play a key role in a broad range of applications, including medicine and forensics. One of the most important of these applications is the diagnosis of genetic disorders. Occurrence of tandem repeats and tandem repeat changes in the human genome have been associated with Huntington's disease, myotonic dystrophy, Friedreich's ataxia, multiple sclerosis, Alzheimer's disease, schizophrenia and cancer.

The invention also allows the evaluation of overall sequence homology, or to detect the presence of a known genetic signature in a sequence. Similarities between sequences allow scientists to infer the function of newly sequenced genes, predict new members of gene families and explore evolutionary relationships. Since whole genomes of many species have been sequenced, sequence similarity searching and pattern identification can be used to predict the location and function of protein-coding and transcription-regulation regions in genomic DNA.

Prior methods have assigned complex numbers to DNA symbols and have computed the periodicity transform based on the sequence of complex numbers. These methods have several disadvantages that include (a) symbol bias, which results in missed detections of some repetitive structures, (b) lack of a post-processing stage for removal of redundant and insignificant repeats and (c) the absence of a strategy for identification of indels.

The present invention addresses all of the above deficiencies. The present invention maps symbols to quaternions, which are four dimensional hypercomplex numbers. This mapping results in an enhanced, symbol-balanced sensitivity of the transform to symbol patterns, in particular DNA and RNA patterns, and an unambiguous threshold selection criterion. Computational efficiency of the transform is further improved, and coupling of the computation with the period value is removed, thereby facilitating parallel implementation of the algorithm. Additionally, a post-processing stage is inserted into the algorithm, enabling unambiguous display of results in a convenient graphical format.

The present invention offers numerous advantages over prior methods. First, replacement of the complex number set in the DNA symbol mapping with the set of quaternions results in a periodicity transform that detects all repetitive structures. The quaternionic approach can be used via the use of the quaternionic Fourier transform to improve spectral methods. Moreover, the computational complexity of the approach is further reduced by a factor equal to the pattern length, a post-processing stage that reduces repeat redundancy is also provided, and a strategy for identification of indels is implemented and discussed.

### Overview of the Methods of the Present Invention

The present invention provides a unique and highly effective method of detecting and outputting tandem repeats in a sequence of symbols. In embodiments, the first step of the method is to identify a sequence of symbols of interest, e.g., a DNA molecule of interest, and map each of the symbols (e.g., nucleotides) to quaternions. For example, if the sequence of symbols is a DNA sequence comprising the letters: A (adenine), C (cytosine), G (guanine) and T (thymine), each symbol would be “mapped,” or assigned, to a quarternion. As noted, a quarternion is a hypercomplex number. For example, in embodiments, the letters associated with each of the nucleotides are assigned to quaternions:

A is assigned to qₒ=i+j+k

C is assigned to q₁=i−j−k

G is assigned to q₂=−i−j+k

T is assigned to q₃=−i+j−k,

where i, j and k are defined by the rules indicated in the “Definitions” section, above. Following assignment of the quaternions to each symbol in the sequence, the result is a sequence of quaternions. For example, if the first six symbols in the DNA sequence were TGC TGC, after mapping the sequence would be q₃q₂q₁ q₃q₂q₁.

In embodiments, after the symbols are mapped to quaternions, a quaternionic periodicity transform (“QPT”) is constructed. As explained in more detail below, for an arbitrary N-point sequence of real numbers “x,” where x=x₀, x₁ . . . x_{N-1}, the QPT, evaluated at a single shift (the actual QPT is a weighted sum of all shifts), can be expressed by the following formula:

$${〈 x 〉}_{q} ( 0 ) = \frac{{( α + β - γ - δ )}^{2} + {( α - β - γ + δ )}^{2} + {( α - β + γ - δ )}^{2}}{3} ,$$

where

$$α = \frac{{\overline{R}}_{a}}{\overline{R}} , β = \frac{{\overline{R}}_{c}}{\overline{R}} , γ = \frac{\overline{R} g}{\overline{R}} , δ = \frac{\overline{R} t}{\overline{R}} ,$$

where R̅ is a factor of R, R is the repetition factor (the maximum number of times a pattern is expected to repeat) and R̅ₐ+ R̅_{c}+ R̅_{g}+ R̅ₜ= R̅. The following equation defines the QPT:

$${〈 x 〉}_{( n )}^{\#} = \frac{1}{P} {\sum }_{t = 0}^{P - 1} 〈 x 〉 ( n + t ) .$$

The parameters of the QPT are the period “P,” the repeat number “ R̅” and the threshold “T.” The period P is the number of symbols present in any given repeat. In embodiments, the period is set to be within a certain range, e.g., from 1≦P≦12 or 1≦P≦48. Because the period is a user-defined parameter, it may be set to any range or value appropriate for the analysis.

The repeat number R̅ is the number of tandem repeats that must be present in order to be included in the final tandem repeat tally. The threshold is the maximum number of errors allowed in a particular repeat. For example, if T=0.85, no errors are permitted in patterns 1-4 symbols long, one error is permitted in patterns 5-8 symbols long and 2 errors are permitted in patterns 9-12 symbols long.

Parameters R̅ and T are co-dependent and determine the sensitivity of the detector to approximate and non-adjacent repeats. In general, large values of R̅ and small values of T increase detector sensitivity to moderately dispersed and approximate tandem repeats. For a fixed P, a larger value of R̅ permits evaluation of a longer subsequence, thereby increasing the chances of capturing a dispersed repeat pattern. Smaller values of T permit more symbol errors.

In general, the computation of

$${〈 x 〉}_{( n )}^{\#} = \frac{1}{P} {\sum }_{t = 0}^{P - 1} 〈 x 〉 ( n + t )$$

requires P evaluations of the transform X_{P}^{N}. However, because

$${〈 x 〉}^{\#} ( {n}_{o} ) = {〈 x 〉}^{\#} ( {n}_{o} - 1 ) - \frac{{\vert {X}_{P}^{N} ( {n}_{o} - 1 ) \vert}^{2}}{P} + \frac{{\vert {X}_{P}^{N} ( {n}_{o} + P - 1 ) \vert}^{2}}{P}$$

the complexity can be reduced to a single evaluation of X_{P}^{N}. This can be done first, by computing, <x>^{#}(0), and then, by performing at each data point the update [symbol] x [symbol] ^{#}(n₀). Computation of this update is particularly simple when R̅=2. In this case a match between two subsequent symbols is given by the formula

|X_{P}^{N}(n₀)|²|=a(n₀)a(n₀+P)+b(n₀)b(n₀+P)+c(n₀)c(n₀+P)+d(n₀)d(n₀+P)

where a, b, c, and d are subsequences of x corresponding to the four DNA symbols. In effect, a single point evaluation of the QPT requires four multiplications and five additions of binary numbers regardless of the value of P. Since the computation is identical and independent for each P, the algorithm can be easily implemented in a multi-processor environment. Moreover, as the computation does not depend on the number of repeats detected, it increases linearly with the data size.

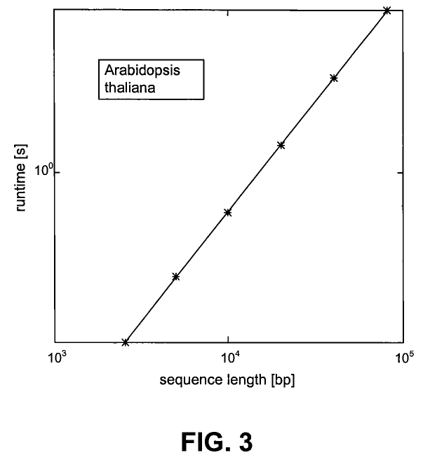

Linear complexity of the QPT computation has been confirmed by a timing experiment, performed on fragments of Arabidopsis thaliana, chromosome 2. Fragment lengths of 2.5K, 5K, 10K, 20K, 40K, and 80K nt (nucleotides), and pattern lengths 1 through 500 were investigated. Experiments were preformed on 2 GHz Pentium PC with 1 GB of memory, running Microsoft Windows XP and Matlab 7.0.1 (R14). Results of the experiment are shown in FIG. 3.

For example, a sequence containing two tandem repeats, is as follows: GTA ACT ACT. Hence, the data can be evaluated over a window of length PR=3*2=6 points. Moving the window in one point increments produces an output sequence 4-points long. The first point of that sequence, at zero shift comes from evaluating the subsequence GTA ACT. Using equations (4), (19) and (20) in the paper (correspondingly, paragraphs 61, 36-39 and 42) yields (3*4)/(3*6*2)=⅓. The denominator, as in (4), is a normalization factor, representing subsequence “energy”, the numerator is the sum of “energies” of three subsequences, GA, TC and AT. This value is the smallest value achievable and indicates a total mismatch (no repetition). Ignoring the next two points, the last point of the sequence, at a shift of 3 points, comes from evaluating the subsequence ACT ACT. Similar computation as above yields (3*12)/(3*6*2)=1. This is the largest value achievable and indicates a perfect match (a repeat of three symbols with no errors). The shift by three indicates the location of the repeated pattern. While this example is to small to talk about errors and partial matches, in general, in longer sequences and patterns the value of the QPT less than 1, but greater than ⅓, indicates an imperfect repeat (an error), or a partial match.

In embodiments, following the construction of the QPT, the QPT of the sequence is computed to determine the number and location of the tandem repeats of the sequence.

In embodiments of the present invention, both the number and location of the tandem repeats are outputted to a computer's memory or to a display screen, or both.

### Periodicity Transform

The periodicity transform was first introduced in the context of speech processing (Sethares and Staley, 1999). It has been subsequently adapted to DNA sequence analysis by Buchner and Janjarasjitt (2003). A brief mathematical description of the formalism is given below.

Detection of P-Periodic Repeats

Factor the data size (existence of such factorization is assumed for notational convenience, but is not required otherwise) N=PR, where P, R ∈ [symbol] are the period and the repetition factor, respectively, and let x be an arbitrary N-point sequence of real numbers, x=x₀, x₁, . . . , x N−1. Define the periodicity transform (PT) by

$$\begin{matrix}{X}_{P}^{R} ( s ) = \frac{1}{R} {\sum }_{k = 0}^{R - 1} x ( s + kP ) , & 0 \leq s < P .\end{matrix} \qquad ( 1 )$$

The periodicity transform identifies all P-periodic subsequences of x. When P is allowed to vary, the periodicity transform identifies all repeats within a given range of P; the positions of those repeats, however, remain unknown (the periodicity transform, as stated here, is identical to the 0th frequency slice through the Zak transform. Zak transform is a time-frequency representation that is intimately related to the Fourier and the short-time Fourier transforms (Brodzik and Tolimieri, 2000). In effect, periodicity transform analysis can be viewed as a special case of time-frequency analysis). In practice, one is often interested in both: detecting occurrence of repetitions and identifying their locations.

Suppose R is not a prime, and choose a factor of R, R̅. In analogy to the short-time Fourier transform, define the short-time periodicity transform (STPT) by

$$\begin{matrix}{X}_{P}^{\overline{R} , R} ( s + rP ) = \frac{1}{R} {\sum }_{k = 0}^{\overline{R} - 1} x ( s + rP + kP ) \\ = [ {X}_{P}^{\overline{R}} ( s ) , {X}_{P}^{\overline{R}} ( s + P ) , … , {X}_{P}^{\overline{R}} ( s + P ( R - \overline{R} ) ) ] ,\end{matrix} \qquad ( 2 )$$

where 0≦r<R− R̅ and 0≦s<P. In contrast to PT, which provides a global characterization of repeats, periodic components identified by the STPT are localized within a segment of length R̅P.

Set n=s+rP. A more robust repeat indicator is obtained, when the STPT is normalized by the R̅P-point segment's ‘power’, and averaged over P shifts, i.e.

$${〈 x 〉}^{\#} ( n ) = \frac{{‖ {X}_{P}^{\overline{R} , R} ( n ) ‖}^{2}}{{‖ x ( n ) ‖}^{2} / \overline{R}} = \frac{{\sum }_{t = 0}^{P - 1} {\vert {X}_{P}^{\overline{R} , R} ( n + t ) \vert}^{2}}{{\sum }_{t = 0}^{\overline{R} P - 1} {\vert x ( n + t ) \vert}^{2} / \overline{R}} . \qquad ( 3 )$$

This indicator is termed the periodicity detector (PD). Since the sequences in the numerator and the denominator of (3), X_{P} ^{R̅,R} and x, are evaluated over P and R̅Ppoints, respectively, hence the normalization factor 1/ R̅.

In the next subsection the properties of PD of a symbolic sequence are investigated, evaluated at a single shift. In preparation, the single shift PD is defined,

$$〈 x 〉 ( n ) = \frac{{\vert {X}_{P}^{\overline{R} , R} ( n ) \vert}^{2}}{{\sum }_{k = 0}^{\overline{R} - 1} {\vert x ( n + kP ) \vert}^{2} / \overline{R}} = \frac{{\vert {\sum }_{k = 0}^{\overline{R} - 1} x ( n + kP ) \vert}^{2}}{\overline{R} {\sum }_{k = 0}^{\overline{R} - 1} {\vert x ( n + kP ) \vert}^{2}} . \qquad ( 4 )$$

Note, that for convenience, contrary to (3), normalization in (4) is performed over a subset of values of x, so that 0≦{x}(n)≦1 for all n. Moreover, if |x(n|²=K for all n for some K ∈ [symbol] , then

$${〈 x 〉}^{\#} ( n ) = \frac{1}{KP} {\sum }_{t = 0}^{P - 1} {\vert {X}_{P}^{\overline{R} , R} ( n + t ) \vert}^{2} = \frac{1}{P} {\sum }_{t = 0}^{P - 1} 〈 x 〉 ( n + t ) . \qquad ( 5 )$$

An abstract periodic symbol detector

Take an arbitrary R̅P-point DNA sequence, x=x₀, x₁, . . . , x _{R̅P-1}, and choose any stride by P R̅-point decimation of x, e.g. x⁰=x₀, x_{P}, . . . , x_{(R-1)P}. Denote by integers R̅ₐ, R̅_{c}, R̅_{g} and R̅ₜ, R̅ₐ+ R̅_{c}+ R̅_{g}+ R̅ₓ= R̅, the count of symbols ‘A’, ‘C’, ‘G’ and ‘T’ in x⁰. Consider an assignment of DNA symbols to arbitrary numbers, e.g.

‘A’ [symbol] _{O0},

‘C’ [symbol] _{O1},

‘G’ [symbol] _{O2},

‘T’ [symbol] _{O3}, (6)

such that O₀≠O₁≠O₂≠O₃ and |O₀|=|O₁|=|O₂|O₃|O|. It follows from (4), that the single shift periodic symbol detector (PSD) of x can then be expressed as

$$〈 x 〉 ( 0 ) = \frac{{\vert α {o}_{0} + β {o}_{1} + γ {o}_{2} + δ {o}_{3} \vert}^{2}}{{\vert o \vert}^{2}} , \qquad ( 7 )$$

where [α, β, γ, δ]=[ R̅ₐ/ R̅, R̅_{c}/ R̅, R̅_{g}/ R̅, R̅ₜ/ R̅]. The detector in (7) is called the abstract periodic symbol detector. The abstract periodic symbol detector needs to satisfy the following conditions: (x [symbol] (0) reaches a minimum when DNA symbols are equally represented in x⁰. In particular, when R̅ is a multiple of four, then [symbol] x [symbol] (0) reaches a minimum for α=β=γ=δ=¼. (x [symbol] (0) reaches a maximum when x⁰ is a string of identical symbols. If α≧β, γ, and δ, then replacement of C, G or T with A in x⁰ increases the value of (x [symbol] (0). (x [symbol] (0) is invariant under permutation of any two symbols.

Complex Assignment of DNA Symbols

Consider an assignment of DNA symbols to complex numbers, e.g.

‘A’ [symbol] p₀=1+i,

‘C’ [symbol] p₁=1−i,

‘G’ [symbol] p₂=−1+i,

‘T’ [symbol] p₃=−1−i. (8)

The complex PSD can then be expressed by the following formula

$$\begin{matrix}{〈 x 〉}_{c} ( 0 ) = \frac{{\vert α {p}_{0} + β {p}_{1} + γ {p}_{2} + δ {p}_{3} \vert}^{2}}{{\vert {p}_{0} \vert}^{2}} \\ = \frac{{( α + β - γ - δ )}^{2} + {( α - β + γ - δ )}^{2}}{2}\end{matrix} \qquad ( 9 )$$

Since for α=β=γ=δ=¼

[symbol]

x [symbol] (0)=0. (10)

and for α=1

[symbol]

x [symbol] _{c}(0)=1, (11)

the complex PSD satisfies conditions 1 and 2. Condition 3, however, is not met since, for example, for α=β=δ=⅓ and γ=0

[symbol]

x [symbol] _{c}(0)= 1/9, (12)

and for α=½, β=⅙, δ=⅓ and γ=0

[symbol]

x [symbol] _{c}(0)= 1/18, (13)

Moreover, since, e.g. for α=γ=½ and β=δ=0

[symbol]

x [symbol] (0)=½, (14)

and for α=δ=½ and β=γ=0

[symbol]

x [symbol] (0)=0, (15)

the condition 4 is violated as well.

In general, the complex PSD given by equations (8-9 ) is variant under exchange of any two parameters, except for the pairs β and γ, and α and δ. In fact, no assignment of type (8) leads to a detector meeting all four conditions of subsection 2.2. In the next section an alternative DNA symbol mapping satisfying all conditions of the abstract PSD will be given.

### The Quaternionic Approach

Real and complex numbers can be viewed as one- and two-dimensional instances of N-dimensional hypercomplex numbers of the form

h=a₀i₀+a₁i₁+a₂i₂+ . . . +a_{N}i_{N}, N ∈ [symbol] υ (0), (16)

where αⱼ ∈ [symbol] 0≦j<N, i₀=1 and iⱼ, 0<j≦N, are symbols called imaginary units. If N=0 then h is real and if N=1 and P₁=−1 then h is complex. The best known hypercomplex numbers, apart from the real and the complex numbers, are the four-dimensional quaternions and the eight-dimensional octonions.

The concept of quaternions was introduced by William Hamilton in 1843 (Hamilton, 1866), who defined a quaternion as a number of the form

q=a+bi+cj+dk, (17)

where a, b, c, d ∈ [symbol] and i, j, k are symbols defined by the following set of rules

i²=j²=k²=−1,

ij=−ji=k, jk=−kj=i, ki=−ik=j, (18)

a is called the real, or scalar, part of q and q−a is called the imaginary, or vector, part of q.
q−a is also known as the pure quaternion.

Applications of quaternions in signal processing include computer vision, robotics (Sommer, 2001), and color and hyperspectral image processing (Sangwine, 1996). For an elementary treatment of quaternions, see (Kantor and Solodovnikov, 1989).

Quaternionic Assignment of DNA Symbols

Consider the assignment of DNA symbols to pure quaternions, e.g.

‘A’ [symbol] q₀=i+j+k,

‘C’ [symbol] q₁=i−j−k,

‘G’ [symbol] q₂=−i−j+k,

‘T’ [symbol] q₃=−i+j−k. (19)

The quaternionic PSD can then be expressed by the following formula

$${〈 x 〉}_{q} ( 0 ) = \frac{{\vert α {q}_{0} + β {q}_{1} + γ {q}_{2} + δ {q}_{3} \vert}^{2}}{{\vert {q}_{0} \vert}^{2}} = \frac{{( α + β - γ - δ )}^{2} + {( α - β - γ + δ )}^{2} + {( α - β + γ - δ )}^{2}}{3} \qquad ( 20 )$$

It is straightforward to verify that the quaternionic PSD satisfies all four conditions of the abstract PSD.

Because of the presence of unequal mutation probabilities of DNA symbols, the invention contemplates appropriately weighting or filtering repeats during post-processing to take such symbol bias into account. An evaluation of these probabilities is not fixed but evolves over sequence length and type. As such, it appears more appropriate to evaluate the probabilities in post-processing.

### Performance Comparison of CPT and QPT

A detailed performance comparison of complex and quaternionic detectors was given in (Brodzik and Peters, 2005). It was shown that use of the CPT might result in missed detection of some nested tandem repeats, while the QPT guaranties detection of all patterns. In an example given in (Brodzik and Peters, 2005), the CPT detected the repeat (AGG)₄(TCC)₄, but missed the repeat (AGG)₄(CTT)₄. Here, the impact of numerical assignment of DNA symbols on threshold selection in the analysis of approximate repeats is discussed. Consider the case of R̅=2, and denote by x [symbol] y a symbol error resulting from replacement of x with y, or y with x. It follows directly from formulas (5) and (9) that for symbol errors of the type c [symbol] g or a [symbol] t, the CPT is

$${〈 x 〉}^{\#} ( 0 ) = \frac{P - ε}{P} \dot{=} {T}_{{c}_{1}} ( P , ε ) . \qquad ( 21 )$$

Conversely, for symbol errors of the type c [symbol] t or a [symbol] g, the CPT is

$${〈 x 〉}^{\#} ( 0 ) = \frac{2 P - ε}{2 P} \dot{=} {T}_{{c}_{2}} ( P , ε ) . \qquad ( 22 )$$

For example, for P=10, the CPT thresholds guaranteeing detection of repeats with at most one and two errors are

T_{c}_{₁}(10, 1)=0.95, T_{c}_{₂}(10, 1)=0.90,

T_{c}_{₁}(10, 2)=0.90, T_{c}_{₂}(10, 2)=0.80,

respectively. Since T_{c2}(10,1)=T_{c1}(10,2), there is no threshold that unambiguously detects all 10 mers with one error or less.

In contrast to the CPT threshold, it follows from formula (20) that the QPT threshold,

$${T}_{q} ( P , ε ) = \frac{3 P - 2 ε}{3 P} , \qquad ( 23 )$$

is identical for all symbol error types.

As an illustration consider two simple VNTR (variable number tandem repeats) of period P=3, containing a single error in the last symbol: ATTATA . . . and ACCACA . . . . Use of (9) and (5) leads to [symbol] x [symbol] (0)=⅔for the first repeat, and [symbol] x [symbol] _{c}^{#(}0)=⅚for the second repeat. Use of (20) and (5) leads to [symbol] x [symbol] (0)= 7/9for either of the two repeats.

Computational Complexity of QPT

In general, the computation in (5) requires P evaluations of the transform X_{P} ^{N̅}. However, since

<x>^{#} (n₀)=<x>^{#} (n₀−1)

$$- \frac{{\vert {X}_{P}^{\overline{N}} ( {n}_{0} - 1 ) \vert}^{2}}{P} + \frac{{\vert {X}_{P}^{\overline{N}} ( {n}_{0} + P - 1 ) \vert}^{2}}{P} , \qquad ( 24 )$$

the complexity of (5) can be reduced to a single evaluation of X_{P} _{N̅}. This can be done first, by computing <x>^{# (}0), and then, by performing at each data point the update (24). Computation of this update is particularly simple when R̅=2. In this case a match between two subsequent symbols is given by the formula

|X_{P} ^{N̅}(n₀)=a(n₀)a(n₀+P)+b(n₀)b(n₀+P) +c(n₀)c(n₀+P)+d(n₀)d(n₀+P), (25)

where a, b, c, and d are subsequences of x corresponding to the four DNA symbols. In effect, a single point evaluation of the QPT requires for multiplications and five additions of binary numbers, regardless of the value of P. Since the computation is identical and independent for each P, the algorithm can be easily implemented in a multi-processor environment. Moreover, as the computation does not depend on the number of repeats detected, it increases linearly with the data size.

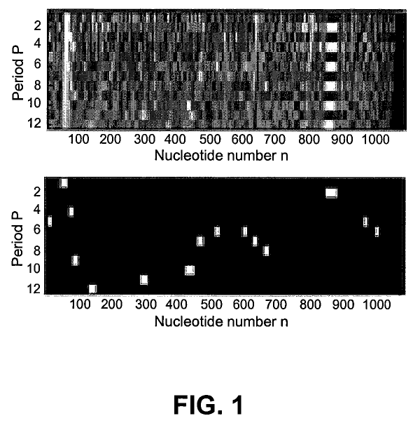

Linear complexity of the QPT computation has been confirmed by a timing experiment, performed on fragments of Arabidopsis thaliana, chromosome 2. Fragment lengths of 2.5K, 5K, 10K, 20K, 40K, and 80K nt, and pattern lengths 1 through 500 were investigated. Experiments were performed on 2 GHz Pentium PC with 1 GB of memory, running Microsoft Windows XP and Matlab 7.0.1 (R14). Results of the experiments are shown in FIG. 1.

Detection of Indels

Several computationally efficient strategies for identifying indels within the periodicity transform framework are available. We briefly describe, using an example, only the simplest of thee strategies, one that allows to detect isolated indels.

Consider an imperfect 4-periodic sequence

| | | | | | |
|---|---|---|---|---|---|
| x: | abcd | abcd | xxx | abcd | abcd, |

where x denotes an arbitrary letter. Suppose the subsequence xxx is the pattern abcd with one of the letters deleted. This deletion can be easily identified using previously computed values of the periodicity transform. For example, when xxx is acd, the single symbol detector S (computed via equation (25)) and the perfect pattern detector T (computed via equation (24) and thresholded at 1.0) take values

| x: | abcd | abcd | acd | abcd | abcd |
|---|---|---|---|---|---|
| S: | 1111 | 1000 | 011 | 1111 | ... |
| T: | 1100 | 0000 | 011 | 1... | ... |

Now, take xxx to be a subsequence that contains only two consecutive matching letters (therefore containing an error and a deletion), for example dbc. Then

| x: | abcd | abcd | dbc | abcd | abcd |
|---|---|---|---|---|---|
| S: | 1111 | 0110 | 000 | 1111 | ... |
| T: | 1000 | 0000 | 000 | 1... | ... |

It can be shown that if x is a sequence of kP−1 letters, then S contains exactly P consecutive zeros and T contains exactly 2P−1 consecutive zeros if and only if an arbitrary repetitive pattern contains a single deletion between any two matching patterns. Similarly, if x is a sequence of kP+1 letters, then S contains exactly P or P+1 consecutive zeros and T contains exactly 2P−1 or 2P consecutive zeros if and only if an arbitrary repetitive pattern contains a single insertion between any two matching patterns. Since the QPT detects all perfect repeats, gaps of P and P+1 zeros between consecutive matching patterns can be easily identified in the QPT plot.

### EXAMPLES

Two repetition-rich well-known sequences, M65145 [GenBank] and U43748 [GenBank], have been investigated. The performance of the QPT has been compared with the performance of the Tandem Repeat Finder (TRF, Benson, 1999) and the Spectral Repeat Finder (SRF, Sharma et al., 2004). The SRF is a recently proposed approach that is a representative of the Fourier space based methods. These methods search for the most frequent repetitions by identifying the dominant frequencies in the short-time Fourier transform spectrum of the sequence. The chief advantage of the SRF over other methods is its ability to detect dispersed repeats. The TRF is a pattern detection method that relies on statistically based recognition criteria. It is the most widely used method for identification of exact and approximate tandem repeats.

The parameters of the QPT are the period P, the repeat number R̅and the threshold T. The period was set to 1≦P≦12 in the first experiment and to 1≦P≦48 in the second experiment, to conform with previous analyses. The remaining two parameters, R̅and T, are co-dependent and determine sensitivity of the detector to approximate and non-adjacent repeats. In general, large values of Rand small values of T increase detector sensitivity to moderately dispersed and approximate tandem repeats. Since the focus of the analysis was on exact and almost exact tandem repeats, R̅=2has been used in both experiments.

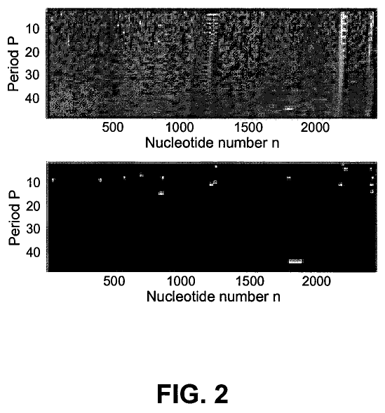

FIGS. 1 and 2 provide graphical display of results. The figures show the raw QPT [computed via equation (3)] and the post-processed QPT. The goal of post-processing is to remove ambiguous and insignificant repeats. These include: (i) repeats with symbol errors exceeding the threshold; (ii) short duration repeats of less than a pre-determined number of DNA symbols;¹ (iii) alias repeats occurring at periods that are multiples of the fundamental period; (iv) repeats that are contained in larger repeats. ¹This step can be removed, if R̅ is allowed to vary in the main stage of the algorithm, so that the product R̅P is not less than the minimum repeat length allowed.

### Example 1

### M65145

In the first experiment an analysis of repeats in the human microsatellite sequence M65145 [GenBank], considered previously in Sharma et al. (2004), was performed. FIG. 1 and Table 1 summarize results of the analysis.

TABLE 1

Exact and approximate tandem repeat patterns of
sequence M65145 detected by QPT and/or TRF. QPT
threshold T = 0.85. TRF version 4.0, parameters:
(match, mismatch, indels) = (2, 7, 7), minimum
alignment score = 20. Symbol substitutions are
denoted by bold face letters. Exact repeats: 2-3,
13. Patterns undetected by TRF: 1, 4, 10-12, 14-15

| | | | | | |
|---|---|---|---|---|---|
| # | Pattern | TRF | P | Position | Copy number |

| | | | | | |
|---|---|---|---|---|---|
| 1 | CCACT | no | 5 | 9:21 | 2.6 |
| | GCACT | | | | |
| 2 | A | yes | 1 | 51:69 | 19.0 |
| 3 | AAGA | yes | 4 | 74:87 | 3.5 |
| 4 | GAAATGATT | no | 9 | 84:101 | 2.0 |
| | GAGGTGATT | | | | |
| 5 | CCTTTGGGGGGT | yes^{a} | 12 | 134:157 | 2.0 |
| | CCTCTGTGGGGT | | | | |
| 6 | ATTGGAGTTTC | yes^{a} | 11 | 293:316 | 2.2 |
| | TTTGGGGTTTC | | | | |
| 7 | GAGGGGTATC | yes^{a} | 10 | 431:458 | 2.8 |
| | TGGGGGTATC | | | | |
| 8 | GGCCCCT | yes | 7 | 467:486 | 2.9 |
| | GTCCCCT | | | | |
| 9 | CTGGCC | yes | 6 | 521:536 | 2.7 |
| | GTGGCC | | | | |
| 10 | TTCCTC | no | 6 | 607:621 | 2.5 |
| | TGCCTC | | | | |
| 11 | TTGGGGG | no | 7 | 638:652 | 2.1 |
| | GTGGGGG | | | | |
| 12 | GCTCTCTG | no | 8 | 672:688 | 2.1 |
| | GCTTTCTG | | | | |
| 13 | GT | yes | 2 | 860:895 | 18.0 |
| 14 | GCTGC | no | 5 | 977:988 | 2.4 |
| | TCTGC | | | | |
| 15 | TCAGCC | no | 6 | 1012:1023 | 2.0 |
| | TGAGCC | | | | |

^{a}Different patterns and/or different copy numbers were obtained by TRF, due to lower threshold used by TRF.

The QPT threshold was set to T=0.85, and the minimum repeat length (P×copy number) was set to 12. The value of the threshold permits no errors in patterns one to four symbols long, one error in patterns five to eight symbols long, and two errors in patterns nine to 12 symbols long (from (23), ε=└L0.225P┘ for T=0.85).

The QPT identified 15 tandem repeats, 3 exact and 12 approximate. The periods of the detected repetitions included all values in the range tested, except for P=3. Among the approximate repeats, repeats #4, #5, #6 and #7 had double letter substitutions. The remaining approximate repeats had a single letter substitution. None of the detected repeats contained indels.

Two of the exact repeats, the singleton A at position 51:69, and the doublet GT at position 860:895, contained a large number of copies, and produced clearly visible in the raw transform plot aliases at multiples of the fundamental period (P≧2 in case of the singleton, and P=2, 4, 6, 8, 10 and 12 in the case of the doublet; FIG. 2). These aliases have been removed in the post-processing stage. Moreover, shorter repeats contained within longer repeats have been removed. While the last step is justified by the need to remove ambiguous and/or insignificant repeats, it may also have the undesirable effect of removing exact repeats masked by approximate repeats. An analysis of the M65145 [GenBank] sequence at threshold T=1.0 revealed presence of exact repeats TGGCCG at position 522:533 and GGGGTATCTG at position 433:452. These repeats have been removed in the post-processing stage of the approximate repeat analysis, as they were contained by repeats #9 and #7, respectively.

The TRF algorithm (Benson's on-line code; version 4.00) was tested with the following parameter settings: (match, mismatch, indels)=(2,7,7), minimum alignment score=20. TRF identified nine repeats. Three of these repeats were identical to the QPT repeats #2, #3 and #13. Out of the remaining six repeats identified by TRF, three were nearly identical to the QPT repeats #6, #8 and #9 (they was shifted with respect to the QPT repeats by a single base (#6, #9) or by a double base (#8) and shorter than the QPT repeats by two bases), two were overlapping repeats of the pattern GGGGTATCTG (433:456 and 433:468), essentially a permutation of the QPT repeat #7, and one was a repeat of the pattern TGACCTTTGGGG, coinciding with and similar to the QPT repeat #5 (131:168). The difference in the pattern content and in the length of repetition in the last two cases resulted from lower threshold used by the Benson code, which allowed detection of patterns having a higher number of errors. The TRF missed the QPT repeats #1, #4, #10-12, and #14-15 (one of the reviewers was able to obtain one more QPT repeat, #11, running the TRF with a minimum alignment score=15). The SRF of Sharma identified only 2 out of the 15 QPT tandem repeats, #5 and #15.

### Example 2

### U43748

In the second experiment an analysis of exact and almost exact repeats in the human frataxin gene sequence U43748 [GenBank], analyzed previously by Benson, was performed. FIG. 2 and Table 2 summarize results of the analysis.

TABLE 2

Exact and approximate tandem repeat patterns of
sequence U43748 detected by QPT and/or TRF. QPT
threshold T = 0.9. TRF version 4.0, parameters:
(match, mismatch, indels) = (2, 7, 7), minimum
alignment score = 30. Symbol substitutions are
denoted by bold face letters. Exact repeats: 4,
13-15. Overlapping pattern groups: (5, 6), (8, 9),
(10, 11), (12, 13), (15, 16, 17, 18). Patterns
undetected by TRF: 1-4, 6, 8, 10, 12

| | | | | | |
|---|---|---|---|---|---|
| # | Pattern | TRF | P | Position | Copy number |

| | | | | | |
|---|---|---|---|---|---|
| 1 | CAACCAAT | no | 8 | 31:49 | 2.4 |
| | NAACCAAT | | | | |
| 2 | GTTTAGAA | no | 8 | 379:395 | 2.1 |
| | TTTTAGAA | | | | |
| 3 | GCGGCCA | no | 7 | 561:574 | 2.0 |
| | GTGGCCA | | | | |
| 4 | GGCCCA | no | 6 | 688:700 | 2.2 |
| 5 | GCCGCGGGCCGCAC | yes | 14 | 822:854 | 2.4 |
| | GCCGNGGGCCGCAC | | | | |
| 6 | GGCCGCA | no | 7 | 842:860 | 2.7 |
| | CGCCGCA | | | | |
| 7 | TGTGTGTGTC | yes | 10 | 1199:1221 | 2.3 |
| | TGTGTGTATC | | | | |
| 8 | CGTGTGTGT | no | 9 | 1228:1246 | 2.1 |
| | TGTGTGTGT | | | | |
| 9 | GT^{a} | yes | 2 | 1229:1249 | 10.0 |
| 10 | AGGAAGG | no | 7 | 1773:1788 | 2.3 |
| | CGGAAGG | | | | |
| 11 | As in Benson | yes | 44 | 1780:1878 | 2.3 |
| | 1999 | | | | |
| 12 | TACTAAAAAA | no | 10 | 2154:2173 | 2.0 |
| | TACAAAAAAA | | | | |
| 13 | A | yes | 1 | 2167:2184 | 18.0 |
| 14 | AAG | yes | 3 | 2183:2211 | 9.7 |
| 15 | AAT | yes^{b} | 3 | 2375:2388 | 4.7 |
| 16 | AATAATAATA | yes^{b} | 10 | 2378:2399 | 2.2 |
| | AATAAAAATA | | | | |
| 17 | AATAATAAATAAA | yes^{b} | 13 | 2381:2407 | 2.1 |
| | AATAAAAAATAAA | | | | |
| 18 | TAAAAAT | yes^{b} | 7 | 2390:2408 | 2.7 |
| | AAAAAAT | | | | |

^{a}Repeat string contains a deletion between nt 1236 and 1237.
^{b}Different patterns and/or different copy numbers (and a different pattern length in case of repeat 18) were obtained by TRF, due to lower threshold used by TRF.

The QPT threshold was set to T=0.9, the period range was set to 1≦P≦48, and the minimum repeat length was set to 12. The QPT threshold allowed for an occurrence of one symbol error for 6≦P≦13, two symbol errors for 14≦P≦19, three symbol errors for 20≦P≦26, four symbol errors for 27≦P≦33, five symbol errors for 34≦P≦39 and a six symbol errors for 40≦P≦46 (from (23), ε=└0.15 P┘ for T=0.9). The QPT found 18 patterns, 4 exact ones and 14 approximate. The pattern periods were: 1, 2, 3, 6, 7, 8, 9, 10, 13, 14 and 44. Twelve of the 18 patterns belonged to one of five groups of overlapping repeats (see caption of Table 2). All approximate repeats had a single letter substitution, except for pattern #9, which had a single deletion, and pattern #11 (a 44 mer), which had six letter substitutions.

The TRF code (match, mismatch, indels)=(2,7,7) was tested in two settings of the TRF minimum alignment score: 50 and 30. The first setting produced four patterns: 3 mer (#14), 13 mer (#17), 14 mer (#5) and 44 mer (#11). Except for the 13 mer, these patterns were described previously in (Benson, 1999). The second setting produced 12 patterns (which included the previous 4 patterns). Out of these 12 patterns, 6 met the error criterion of the QPT, and were identical to the patterns detected by the QPT. Out of the remaining six repeats, five repeats (#15-18) were similar to repeats detected by the QPT. Among those were the 10 mers AAAAATAATA (TRF) and AATAATAATA (QPT), occurring at overlapping regions 2372:2399 and 2378:2399, respectively. Since the TRF allowed for more errors than the QPT, it started counting repeats six bases earlier than the QPT, which resulted in a different pattern. One pattern, the 12 mer AAGGCACGGGCG, identified by TRF, was undetected by QPT, due to the presence of two deletions.

Out of the 18 patterns identified by the QPT, 8 were undetected by the TRF. These included the 6 mer GGCCCA, the 7 mers GCGGCCA, GGCCGCA and AGGAAGG, the 8 mers CAACCAAT and GTTTAGAA, the 9 mer CGTGTGTGT and the 10 mer TACTAAAAAA. Four more repeats (two of them overlapping with the reported repeats) have been found testing the TRF code with the lowest minimum alignment score allowed, 20, all of them, however, had a higher percentage of errors than allowed in the experiment. No dispersed repeats were found in the sequence using either the SRF or the QPT.

### SUMMARY

The approach of the present invention incorporates several features that significantly improve performance and extend the utility of the periodicity transform. These advantages include: invariance to symbol permutation, a convenient for multiple queries two-stage data processing scheme, a factor of P computational efficiency improvement, removal of aliases and an efficient mechanism for dealing with isolated indels.

The standard mapping of DNA symbols to complex numbers has been replaced with a mapping of DNA symbols to quaternionic numbers. This mapping removes preferential treatment of DNA symbols occurring in the complex mapping, and guarantees detection of all patterns, including complex, multi-period patterns, described by Hauth and Joseph (2002). In approximate repeat analysis, the quaternionic mapping leads to a threshold that is unambiguously coupled with the pattern length and the number of errors, and that is independent of pattern and error type. Numerical experiments have shown that the QPT is competitive with the industry standard, TRF, in that it detects patterns that are missed by TRF.

The QPT approach consists of two stages of processing. In the first stage all repeats are detected. In the second stage repeats that have high number of errors, are short, or ambiguous, are removed. The choice of two stage processing was dictated by computational convenience, as the number of relatively expensive computations of the second stage can be reduced by operating on a much smaller subset; such approach, however, has a number of other advantages as well. First, graphical display of results of both stages of the computation is produced, allowing visual evaluation. Second, since results of the first stage of the computation are stored, the second stage can be conveniently repeated for a different choice of pattern selection parameters, and perhaps for a subset of data, at a later time, without repeating the entire computation. Apart from graphical display, the algorithm produces a numerical listing of results.

One of the chief inconveniences of the original approach was the appearance of aliases in the periodicity transform plot. Here, repeats that occur at identical locations for multiples of the fundamental period, and repeats that are entirely contained in larger repeats, are removed. Repeats that partly overlap are kept, as such repeats cannot be uniquely ordered in importance strictly based on mathematical criteria (particularly in the presence of symbol errors), and as decision about which of these repeats is biologically relevant is application dependent.

The main computational burden is carried by the first stage of the algorithm. The complexity of the computation is reduced by a factor of P in comparison with the direct approach. In contrast to most reports on DNA repeat detection methods, which either provide only rough estimates of complexity, or give timing results of selected experiments, complexity of the QPT can be evaluated exactly. It was shown that computational complexity of the QPT is strictly linear with the data size, for a fixed range of period sizes, and that it requires only a single evaluation of a symbol match at each point (which consists of five additions and four multiplications in the current implementation of the algorithm). Moreover, since the evaluation is performed independently and identically for all period values, the algorithm can be easily implemented in a multi-processor environment.

An important discovery of this work was the finding that the first stage of processing produces intermediate results that can be used to detect indels. A scheme for identification of single indels has been implemented.

The breadth and scope of the present invention should not be limited by any of the above-described exemplary embodiments, but should be defined only in accordance with the following claims and their equivalents.

What is claimed is:

1. A method of detecting and outputting tandem repeats in a sequence of symbols comprising:
(a) mapping the symbols to quaternions;
(b) constructing a Quaternionic Periodicity Transform (QPT);
(c) computing the QPT of the sequence to determine the tandem repeats of the sequence;
(d) post-processing of the QPT;
(e) outputting a list of tandem repeats obtained from step d) to a computer's memory.

2. The method of claim 1, wherein the tandem repeats consist of two or more adjacent copies of an arbitrary sequence of the symbols.

3. The method of claim 1, wherein the sequence of symbols is a DNA sequence, wherein each type of nucleotide is represented by a unique symbol.

4. The method of claim 3, wherein the unique symbols are A, T, G and C.

5. The method of claim 1, wherein the sequence of symbols is an RNA sequence, wherein each type of nucleotide is represented by a unique symbol.

6. The method of claim 5, wherein the unique symbols are A, U, G and C.

7. The method of claim 1, wherein the mapping comprises assigning each of the symbols to a pure quaternion that results in invariance of the QPT to symbol permutations.

8. The method of claim 1, wherein the length of the tandem repeats is equal to or longer than one symbol.

9. The method of claim 2, wherein the adjacent copies of symbols are of equal length.

10. The method of claim 2, wherein the adjacent copies of symbols are of unequal length.

11. The method of claim 1, wherein the post-processing of the QPT comprises removing tandem repeats that are either short, ambiguous, or contain a high number of errors.

12. The method according to claim 11, wherein the removed tandem repeats include repeats with symbol errors exceeding a pre-determined threshold, short duration repeats of less than a pre-determined number of symbols, alias repeats occurring at periods that are multiples of the fundamental repeats and repeats that are contained in larger repeats.

13. The method of claim 1, further comprising displaying the list of tandem repeats in graphical form.

14. The method of claim 1, wherein the tandem repeats are exact repeats.

15. The method of claim 1, wherein the tandem repeats are approximate repeats.

16. The method of claim 1, wherein the tandem repeats are nested tandem repeats.

17. A method of detecting and outputting tandem repeats in a sequence of symbols comprising:
(a) mapping the symbols to quaternions to obtain a numerical sequence;
(b) applying periodicity transform on a subsequence of the numerical sequence at each position of the sequence to generate the closest periodic sequence to the subsequence;
(d) repeating step b) for each portion of the sequence and selecting repeats that satisfy predetermined thresholds;
(e) removing from the selected repeats those repeats with symbol errors exceeding a pre-determined threshold, short duration repeats of less than a pre-determined number of symbols, alias repeats occurring at periods that are multiples of the fundamental repeats and repeats that are contained in larger repeats;
(f) outputting sequence repeats, the number of repeats, the positions of the repeats and the length of the repeats to a computer's memory; and
(g) displaying the result from step f) in a graphical format.

18. The method of claim 17, wherein the sequence of symbols is a DNA sequence, wherein each type of nucleotide is represented by a unique symbol.

19. The method of claim 17, wherein the sequence of symbols is an RNA sequence, wherein each type of nucleotide is represented by a unique symbol.
